(19) United States
(12) Patent Application Publication
(10) Pub. No.: US 2011/0275989 A1
(43) Pub. Date: Nov. 10, 2011

(54) HERMETICALLY SEALED MICRO DISPENSING DISPOSABLE MEDICAL DEVICE

(75) Inventors: Venkata Surya Jagannath Yedida, Andhra Pradesh (IN); Venkata Satyanarayan Nadupalli, Andhra Pradesh (IN)

(73) Assignee: M/S. REEVAX PHARMA PRIVATE LIMITED, Andhra Pradesh (IN)

(21) Appl. No.: 13/143,691

(22) PCT Filed: Nov. 20, 2009
(86) PCT No.: PCT/IN2009/000674

§ 371 Date: Jul. 7, 2011

(30) Foreign Application Priority Data
Jan. 7, 2009 (IN) 40/CHE/2009

(51) Int. Cl. A61M 37/00

(52) U.S. Cl. 604/82; 604/244

(57) ABSTRACT

A hermitically sealed micro dispensing disposable medical device made with metallic or glass or ceramic or polymeric ultra thin foils or membranes, with independent puncturing mechanisms for isolating and stabilizing the desired liquids until the device is ready for use which comprises of (a) canula (b) hub, (c) jacket and (d) stub characterized in that cannula consists of flexible, bendable, fixable capillary tubing, and is designed to bendable to any angle up to forty five degrees as measured from the vertical, the functionality for flexible bent, hub consists of screwed top end hub tube with a screw thread on the outside which mates with the screw end of the cannula to provide a flow control orifice system for the preferred embodiment, ultraviolet light source, preferably an uv led, is embedded at the circumference end of the hub. under the uv led, a battery power source is located, jacket is made up of flexible polymer tubing made from a material like polypropylene, polyethylene, PTFE, PBT etc or a combination thereof and preferably be transparent or translucent with sufficient structural strength, stub consists of an auxiliary canister made similarly like the main canister, auxiliary canister is filled with a desired auxiliary liquid.

### FIELD OF THE INVENTION

The present invention relates to hermitically sealed micro dispensing disposable medical device.

### BACKGROUND OF THE INVENTION

Generally medical liquids like lotions, vaccines, adhesives, sterile fluids and other pharmaceutical, bio-medical ingredients are filled in a specifically designed container and drawn at the time of use wider normal room temperature and generally under non-sterile conditions with uncontrolled means of delivery.

Unlike above, this device, hermitically sealed micro dispensing disposable medical device, having various integrated, concise and practical units and systems in-built can alleviate the sterility, temperature stability and controlled fluid delivery problems faced every day by various medical and pharmaceutical professionals. The main disadvantages of generally medical liquids like lotions, vaccines, adhesives, sterile fluids and other pharmaceutical, bio-medical ingredients are filled in a specifically designed container and drawn at the time of use under normal room temperature and generally under nonsterile conditions with uncontrolled means of delivery.

Unlike above, this device, hermitically sealed micro dispensing disposable medical device, having various integrated, concise and practical units and systems in-built can alleviate the sterility, temperature stability and controlled fluid delivery problems faced every day by various medical and pharmaceutical professionals.

Therefore, in view of the disadvantages stated above, the main objective of the present invention is the micro delivery system dispenses the required quantity of medically useful fluid with ease of simple, hand operable and flow controllable manner.

This invention relates to a disposable device for delivering one or more liquid products simultaneously and has particular application in areas where controlled release of one or more liquids is required, the product being dispensed, such as in an air freshener or insecticide or medicine or adhesive, on to a surface or place like skin, tissue, metal parts or sections, or into a flow of other dispersing liquids such as in analytical systems, production lines, medicine application sites, manufacturing equipment parts, surfaces etc.

The main object of the present invention is to provide a dispensing device that is particularly concerned with delivering products, such as adhesives, glue formulations, pharmaceutical ingredients, medicines, chemicals, perfumes, surfactants, bleaches, disinfectants and combinations thereof, particularly in the form of liquids, solutions, dispersions, suspensions.

Another object of the present invention is to provide a micro cannula that is particularly concerned with delivering liquid products, to a site where precise location is of concern. Yet another object of the present invention is to provide a flexible, bendable and fixable cannula to dispense liquids in hard to reach application sites such as corners, bends, crevices, curved surfaces, small tunnels or holes.

One object of the current invention is to provide a flow control orifice system to dispense precise amount of liquid at the site of application, as excess application of the liquids can result in dangerous side effects in case of medicines and pharmaceutical ingredients and unwanted reactions in case of chemicals and reagents and slippage and wastage in case of manufacturing or co-bonding in case of adhesives.

Still another object of the present invention is the ability of the device to provide a in-built visible light source to illuminate the site of application thus assisting the application to be executed even in semi-darkened or completely darkened sites, places, crevices, corners, bends, tunnels, holes, surfaces etc.

Further object of the present invention is the ability of the device to provide a in-built ultraviolet light source to illuminate the application site that will assist to illuminate various surfaces that can be seen better under ultraviolet light.

Yet further object of the present invention is to provide in-built ultraviolet light illumination for the ability to bring about in some cases, in-situ chemical, photochemical, reactions, changes, modifications to the liquid products that are being applied or just applied at the application site.

Yet another object of the present invention is to provide three filter cartridges in the device to bring about various in-situ chemical and on-line physical changes to the liquids that are being dispensed.

Still further object of the invention is to provide for a jacket that has been embedded or coated or sprayed along the inside wall, with chemical moieties like polymers, chemicals, medicines, pharmaceutical ingredients, adhesives, glues or combinations thereof, physical units like powders, nano particles, micro particles or combinations thereof to provide for various chemical, physical, photochemical reactions, modifications, reactions, changes to the liquids that are being dispensed using the device.

Yet another object of the invention is to provide two inert canisters to store two liquids under positive pressure for quick and efficient dispensing of the liquids. This aspect of the invention also provides the ability to isolate the said liquids from the atmosphere, moisture, radiation, dust, gases till the time of application.

Yet further object of the invention is to provide for independent puncturing mechanisms for each of the canisters storing the liquids thus providing the invention the ability to selectively dispense one of the two liquids or simultaneously dispense the two liquids together at the site of application.

These and other objects of the present invention will become apparent to those skilled in the art as the description thereof proceeds. According to the present invention there is provided a hermitically sealed micro dispensing disposable medical device made with metallic or polymeric ultra thin foils or membranes, with independent puncturing mechanisms for isolating and stabilizing the desired liquids until the device is ready for use which comprises of (a) canula (b) hub, (c) jacket and (d) stup characterized in that:
cannula consists of flexible, bendable, fixable capillary tubing, and is designed to bendable to any angle up to forty five degrees as measured from the vertical, the functionality for flexible bent;
hub consists of screwed top end hub tube with a screw thread on the outside which mates with the screw end of the cannula to provide a flow control orifice system for the preferred embodiment, ultraviolet light source, preferably an uv led, is embedded at the circumference end of the hub, under the uv led, a battery power source is located;
jacket is made up of flexible polymer tubing made from a material like polypropylene, polyethylene, PTFE, PBT etc or a combination thereof and preferably be transparent or translucent with sufficient structural strength;
stub consists of an auxiliary canister made similarly like the main canister, auxiliary canister is filled with a desired auxiliary liquid. A staggered three filter cartridge unit independently impregnated with silver nano or micro particles to assist in sterilization of the said liquids.

A canister is filled with positive pressurized liquid made with metallic or polymeric ultra thin foil with a thickness profile to bring about precise location puncturing. A jacket tubing is coated or sprayed or embedded inside with chemical moieties or physical units or a combination thereof to assist in various aspects like stabilization, initiations, reactions, catalyze, sterilization and combination thereof in conjunction with the said liquids.

A staggered three filter cartridge unit is independently impregnated with various chemical moieties or physical units or a combination thereof to assist in various aspects like stabilization, initiations, reactions, catalyze, sterilization, micro filtering and a combination thereof in conjunction with the said liquids.

A staggered three filter cartridge unit is independently impregnated with silver nano or micro particles to assist in sterilization of the said liquids. A flexible, bendable, fixable cannula system with a built-in liquid flow control orifice unit.

A built-in visible spectrum light source precisely directed towards the application site. A built-in ultraviolet light source to precisely directed towards the application site.

### DESCRIPTION OF THE ACCOMPANYING DRAWING

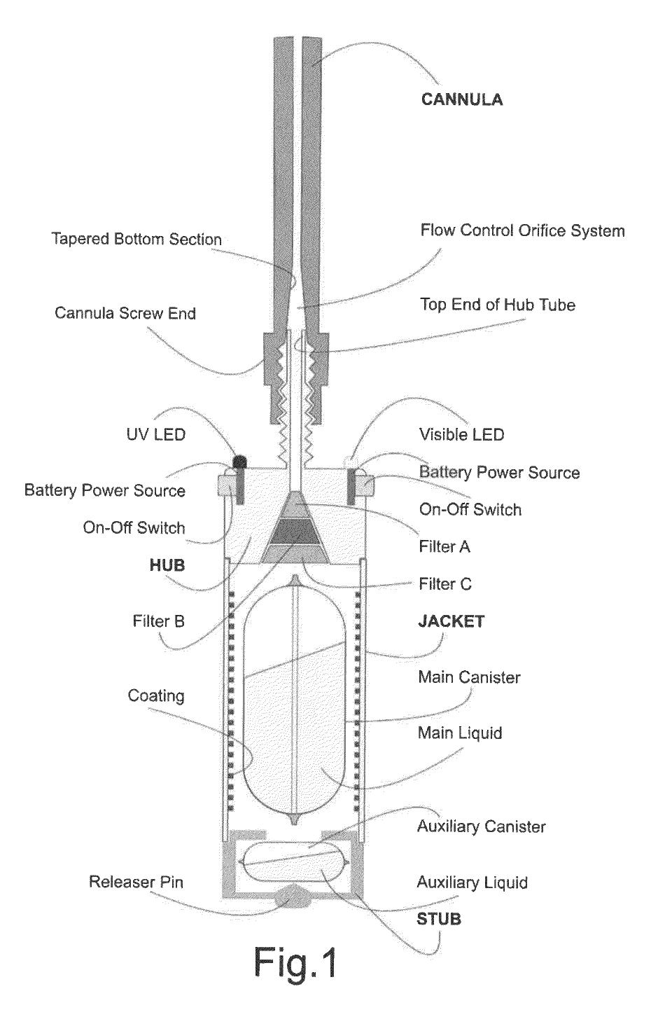

The present invention will be described with simplicity and specificity and clarity with reference to drawing given under FIG. 1.

The preferred embodiment consists of four components, namely, CANNULA, HUB, JACKET and STUB.

### CANNULA Component:

The cannula consists of flexible, bendable, fixable capillary tubing generally made of metals like aluminum, copper, stainless steel etc or polymers like polypropylene, polyethylene, polyester, PTFE etc or a combination thereof. The cannula is designed to bendable to any angle up to forty five degrees as measured from the vertical.

The functionality for flexible bent cannula is bought out by using concentric tubes of varying flexibilities and rigidities as required by the application. Alternatively, a non-elastic flexible and malleable fine wire or tubing made out of a polymer or a metal is inserted into the cannula so that it will effect the required physical characteristic of the cannula.

Using finger tips, the cannula can be bent and fixed at the desired angle up to forty five degrees. The cannula contains a tapered bottom section and a screw thread on the inside.

When the HUB and the CANNULA are screwed together the top end of Hub tube ends in the tapered bottom section of the CANNULA providing a flow control orifice system.

By tightening the CANNULA on the HUB will decrease the diameter of the top end Hub tube thus decreasing the flow of liquid though the cannula.

Alternatively by loosening the CANNULA on the HUB will increase the top end Hub tube diameter thus increasing the flow of liquid through cannula.

Thus a fine and continuous control of the flow of liquid, is obtained during the operation of the preferred embodiment.

### HUB Component:

The HUB consists of screwed top end Hub Tube with a screw thread on the outside which mates with the screw end of the CANNULA to provide a flow control orifice system for the preferred embodiment.

Ultraviolet light source, preferably an UV LED, is embedded at the circumference end of the HUB. Under the UV LED, a battery power source is located which in turn is connected to an On Off switch which is available on the side of the HUB.

Similarly, on the radially opposite location of the HUB, a visible light source, preferably an LED, is embedded along with a battery power source as well as a On-Off switch.

Alternatively two UV LEDs and two visible LEDs can be embedded into the HUB at alternate locations, to provide even better illuminations at the site of application. The visible spectrum LED illuminates the site of application for offering better visibility.

The UV LED is used for initiation, continuation, completion reactions of certain polymers, monomers, catalysts and other chemical entities at the application site.

The UV LED also assists in making visible some surfaces, objects etc that are not normally visible under normal lighting conditions.

Both light sources can independently operated by using their respective On-Off switches.

The HUB has three staggered filter layer cartridge system (Filter A, Filter B, Filter C) embedded a the bottom in-line with the flow of the liquid.

Each of the filters A, B & C made up of a polymer matrix embedded with various chemical or physical ingredients or moieties like powders, micro particles, nano particles, enhancers, stabilizers, catalysts, initiators, sterilizers or combinations thereof.

### JACKET Component:

The jacket is made up of flexible polymer tubing made from a material like polypropylene, polyethylene, PTFE, PBT etc or a combination thereof and preferably be transparent or translucent with sufficient structural strength.

The inner surface of the jacket tubing has a coating that is coated or sprayed or embedded with chemical moieties or physical entities like nano or micro particles, that assist in various aspects like stabilization, initiation, reaction, catalyzation or a combination thereof.

The coating is preferably applied, in the form of liquid crystals, nano particles, micro particles, surfactants, waxes etc or a combination thereof.

This coating is quickly reacts or mixes or solubulizes or a combination thereof with the liquids that fill the jacket at the time of use of the preferred embodiment.

The JACKET component contains the main canister that is preferably made out of micro thin metal foil like aluminum, copper, stainless steel or a combination thereof. The canister can also be made out of various polymers like polypropylene, polyethylene, PTFE, PBT or a combination thereof or with materials like glass or ceramic.

The main canister is made in such a way when punctured using external pressure, the location of puncture is at the bottom of the canister. This requirement is brought out by manufacturing the canister using varying thickness profile that has the lowest thickness at the bottom.

The main canister is filled fully or partially with the desired liquid that needs to be dispensed. This liquid is filled under positive pressure in the canister.

The positive pressure in the canister facilitates the ejection of liquid that is filled inside into the JACKET in the event of puncturing of the canister due to external pressure by piercing or twisting or elongating or compressing or surface tampering or a combination thereof.

The main canister is punctured by applying external pressure diametrically over the JACKET using fingers or any other another external device.

The main canister also assists in isolating the liquid from atmosphere, moisture, radiation, dust, gases till the time of application.

### STUB Componet:

The STUB consists of an auxiliary canister made similarly like the main canister. Auxiliary canister is filled with a desired auxiliary liquid. The STUB provides for a mechanism that facilitates puncturing of auxiliary canister to bring out the auxiliary liquid that is filled inside the auxiliary canister.

External pressure is applied by pressing with a finger on the bottom surface of the STUB the releaser pin location.

### DETAILED DESCRIPTION OF THE PREFERRED EMBODIMENT

The preferred embodiment can be utilized as a disposable device micro dispensing of one or two liquid products. The choice of using the main liquid in the main canister with or without the auxiliary liquid in auxiliary canister can be made before the start of the usage of the device.

If the auxiliary liquid is not to be utilized in the desired application, only the main canister is punctured using firm external pressure diametrically on the JACKET of the preferred embodiment.

The main liquid from the main canister flows into the JACKET on breaking or crushing or puncturing of the main canister. If the auxiliary liquid is desired in the application, the release pin is pushed at the bottom of STUB to puncture the auxiliary canister to release the auxiliary liquid into the JACKET.

Both the liquids mix in the JACKET. Mixing of the liquids is accelerated by shaking the device. The liquid will be acted upon the coating available on the inner surface of the JACKET.

The On/Off Switches on the HUB are used as and when required to make available, ultraviolet light and visible light at the time of use of the preferred embodiment. The cannula screw end is tightened or loosened on the HUB as desired to control the flow of the liquid at the application site. The cannula is bent at an angle or left straight as desired.

The cannula is brought near the application site and the JACKET is pressed between two fingers to force the liquid out of the device. As the liquids force out of the cannula they pass through the filters A, B & C situated in the HUB. The chemical moieties or physical entities embedded into the polymer matrix of these filters bring about various chemical or physical reactions, modifications, changes to the liquids that are being dispensed using the device.

The visible spectrum LED assists in dispensing the liquid by the used at precisely at the desired location at the application site. This visible light source can be switched on or off as desired by utilizing the On-Off switch. The ultraviolet LED will assist optionally in showing the surface of the application site that can be seen only in UV light. This UV light source can be switched on or off as desired by utilizing the On-Off switch.

The ultraviolet LED will assist optionally in bringing out various reactions, like cross-linking of monomer liquids that are being applied on the site. Once the application of liquid on the application site is ceased, the visible LED can still be used to check the completion of the work at the application site.

Similarly the ultraviolet LED can be used to continue various reactions, like cross-linking to proceed further, if required by the application.

The device is disposed in a suitable manner once the application is performed to completion.

It will be understood by those skilled in the art that numerous improvements and modifications may be made to the preferred embodiment of the present invention disclosed herein without departing from the spirit and scope thereof.

We shall now describe the present invention by way of illustrations which particularly describes the invention and does not restrict the scope of the present invention.

Product usage in the present embodiment is exemplified in Example 1.

### Example 1

| Main | Auxiliary | | | | |
|---|---|---|---|---|---|
| Canister | Canister | Filter A | Filter B | Filter C | Coating |
| Adhesive | Monomer | Catalyst for | Catalyst for | Particulate | Initiator |
| | | Adhesive | Monomer | Filter | |
| Catalyst | Suspension | initiator for | Initiator for | Particulate | Catalyst |
| Liquid | | Liquid 1 | Liquid 2 | Filter | Stabilzer |
| Medical | Coloring | Silver | Microbial | Particuiate | Cross |
| Glue | Solution | Nano | Filter | Filter | Linking |
| | | Particles | | | Spray |

1. A hermetically sealed micro dispensing disposable medical device made with metallic or glass or ceramic or polymeric ultra thin foils or membranes, with independent puncturing mechanisms for isolating and stabilizing the desired liquids until the device is ready for use comprising (a) cannula (b) hub, (c) jacket and (d) stub characterized in that:
cannula comprising a flexible, bendable, fixable capillary tubing, and is designed to bendable to any angle up to forty five degrees as measured from the vertical, the functionality for flexible bent;
hub comprising a screwed top end hub tube with a screw thread on the outside portion which mates with the screw end of the cannula to provide a flow control orifice system, ultraviolet light source; preferably an UV LED, is embedded at the circumference end of the hub and under the UV LED, a battery power source is located;
jacket is made up of flexible polymer tubing made from a material like polypropylene, polyethylene, PTFE, PBT etc or a combination thereof,
stub comprising an auxiliary canister filled with a desired auxiliary liquid.

2. The hermetically sealed micro dispensing disposable medical device made according to claim 1, further comprising a thickness profile to bring about precise location puncturing.

3. The hermetically sealed micro dispensing disposable medical device according to claim 1, wherein said jacket tubing is preferably transparent or translucent with sufficient structural strength.

4. The hermetically sealed micro dispensing disposable medical device according to claim 1, wherein said jacket tubing coated or sprayed or embedded inside with chemical moieties or physical units or a combination thereof to assist in various aspects like stabilization, initiations, reactions, catalyze, sterilization and a combination thereof in conjunction with the said liquids.

5. The hermetically sealed micro dispensing disposable medical device according to claim 1, further comprising a staggered three filter cartridge unit independently impregnated with various chemical moieties or physical units or a combination thereof to assist in various aspects like stabilization, initiations, reactions, catalyze, sterilization, micro filtering and combination thereof.

6. The hermetically sealed micro dispensing disposable medical device according to claim 1, wherein said flexible capillary tubing is made of metals like aluminum, copper, stainless steel, etc., or glass or ceramic or polymers like polypropylene, polyethylene, polyester, PTFE, etc. or a combination thereof.

7. The hermetically sealed micro dispensing disposable medical device according to claim 1, wherein said cannula is bought out by using concentric tubes of varying flexibilities and rigidities as required by the application.

8. The hermetically sealed micro dispensing disposable medical device according to claim 1, wherein a non-elastic flexible and malleable fine wire or tubing made out of a polymer or a metal is inserted into the cannula so that it will effect the required physical characteristic of the cannula, using finger tips, the cannula is bent and fixed at the desired angle up to forty-five degrees, the cannula contains a tapered bottom section and a screw thread on the inside portion.

9. The hermetically sealed micro dispensing disposable medical device according to claim 1, further comprising a battery power source is connected to an on-off switch available on the side of the hub, and also radially opposite location of the hub, a visible light source, preferably an LED, is embedded along with a battery power source as well as a on-off switch, alternatively two UV LEDs and two visible LEDs are embedded into the hub at alternate locations, to provide better illuminations at the site of application, the hub has three staggered filter layer cartridge system (filter a, filter b, filter c) embedded at the bottom in-line with the flow of liquid.

10. The hermetically sealed micro dispensing disposable medical device according to claim 1, wherein the inner surface of the jacket tubing has a coating that is coated or sprayed or embedded with chemical moieties or physical entities like nano or micro particles, which assists in various aspects like stabilization, initiation, reaction, catalyzation or a combination thereof.

11. The hermetically sealed micro dispensing disposable medical device according to claim 10, wherein the coating is preferably applied in the form of liquid crystals, nano particles, micro particles, surfactants, waxes etc or a combination thereof.

12. The hermetically sealed micro dispensing disposable medical device according to claim 1, wherein said stub provides a mechanism that facilitates puncturing of auxiliary canister to bring out the auxiliary liquid that is filled inside the auxiliary canister, wherein external pressure is applied by pressing with a finger on the bottom surface of the stub the releaser pin location.
